(19) United States
(12) Patent Application Publication
(10) Pub. No.: US 2025/0169788 A1
(43) Pub. Date: May 29, 2025

(54) RADIOGRAPHIC IMAGING CONTROL APPARATUS AND RADIOGRAPHIC IMAGING SYSTEM

(71) Applicant: CANON KABUSHIKI KAISHA, Tokyo (JP)

(72) Inventor: TAKUYA TSUKADA, Chiba (JP)

(21) Appl. No.: 18/952,684

(22) Filed: Nov. 19, 2024

(30) Foreign Application Priority Data
Nov. 27, 2023 (JP) 2023-200147

(51) Int. Cl. A61B 6/00

(52) U.S. Cl. CPC A61B 6/545; A61B 6/542

(57) ABSTRACT

One or more embodiments of a radiographic imaging control apparatus controls radiographic imaging performed by emitting radiation to at least one radiation detection apparatus using at least one radiation generation apparatus from among a plurality of radiation generation apparatuses, and the radiographic imaging control apparatus may include a first acquisition unit configured to acquire information about the radiographic imaging, an analysis unit configured to analyze the information acquired by the first acquisition unit to determine at least whether a target or subject to be examined is present, and a determination unit configured to determine a radiation generation apparatus to be used for the radiographic imaging among the plurality of radiation generation apparatuses, based on a result of analysis performed by the analysis unit, wherein the radiographic imaging control apparatus makes or generates a setting related to the radiation generation apparatus determined to be used for the radiographic imaging.

### CROSS-REFERENCE TO RELATED APPLICATION(S)

This application claims the benefit of Japanese Patent Application No. 2023-200147, filed Nov. 27, 2023, which is hereby incorporated by reference herein in its entirety.

### BACKGROUND

### Field of the Disclosure

One or more features of the present disclosure relate to a radiographic imaging control apparatus that controls radiographic imaging, and a radiographic imaging system.

### Description of the Related Art

A radiographic imaging apparatus that uses a flat panel detector (FPD) made of a semiconductor material has been in widespread use as an imaging apparatus used for a medical image diagnosis and a non-destructive inspection using radiation. Such a radiographic imaging apparatus is, for example in the field or area of medical image diagnosis, used as a digital image capturing apparatus for still image capturing such as radiography and for moving image capturing such as fluoroscopy.

In general, in the field or area of radiographic imaging, an X-ray tube for emitting radiation may be determined to be suitable for an examination, and Japanese Patent Application Laid-open No. 2019-198691 discusses a means for associating an imaging condition and an X-ray tube without manually determining an X-ray tube for emitting radiation at the time of an examination. In the case of the radiographic imaging system discussed in Japanese Patent Application Laid-open No. 2019-198691, to uniquely determine an X-ray tube, imaging information and identification information for uniquely identifying an X-ray tube are associated with each other.

### SUMMARY

According to one or more aspects of the present disclosure, a radiographic imaging control apparatus may control radiographic imaging that is performed by emitting radiation to at least one radiation detection apparatus using at least one radiation generation apparatus from among a plurality of radiation generation apparatuses, and the radiographic imaging control apparatus may include a first acquisition unit that operates or is configured to acquire information about the radiographic imaging, an analysis unit that operates or is configured to analyze the information acquired by the first acquisition unit to determine at least whether a target or subject to be examined is present, and a determination unit that operates or is configured to determine a radiation generation apparatus to be used for the radiographic imaging among the plurality of radiation generation apparatuses, based on a result of analysis performed by the analysis unit, wherein the radiographic imaging control apparatus makes or generates a setting related to the radiation generation apparatus determined by the determination unit to be used for the radiographic imaging. In one or more embodiments, the radiographic imaging control apparatus may include one or more processors that operate to perform or apply the setting related to the radiation generation apparatus on or to the radiation generation apparatus determined by the determination unit to be used for the radiographic imaging.

According to other aspects of the present disclosure, one or more additional control apparatuses and/or radiographic imaging apparatuses or systems and one or more methods are discussed herein. Further features of the present disclosure will become apparent from the following description of exemplary embodiments with reference to the attached drawings.

### BRIEF DESCRIPTION OF THE DRAWINGS

FIG. 1 is a block diagram illustrating an example of a basic configuration of a radiographic imaging system according to one or more embodiments of the present disclosure.

FIG. 2 is a diagram illustrating an example of a configuration of the radiographic imaging system according to one or more embodiments of the present disclosure.

FIG. 3 is a block diagram illustrating a detailed configuration of an optical image analysis unit according to one or more embodiments of the present disclosure.

FIG. 4 is a flowchart illustrating selection processing for a radiation generation apparatus using an optical imaging apparatus according to one or more embodiments of the present disclosure.

FIG. 5 is a flowchart illustrating analysis processing for an optical image acquired from the optical imaging apparatus according to one or more embodiments of the present disclosure.

FIG. 6 is a block diagram illustrating an example of a configuration of a radiographic imaging system according to one or more embodiments of the present disclosure.

FIG. 7 is a flowchart illustrating setting processing for a radiation generation apparatus using an optical imaging apparatus according to one or more embodiments of the present disclosure.

FIG. 8 is a flowchart illustrating analysis processing for an optical image acquired from the optical imaging apparatus according to one or more embodiments of the present disclosure.

### DESCRIPTION OF THE EMBODIMENTS

Hereinbelow, one or more embodiments and/or features of the present disclosure will be described with reference to the attached drawings. Note that configurations illustrated in the following embodiments are merely examples, and the present technique(s)/feature(s) of the present disclosure is/are not limited to the illustrated configurations.

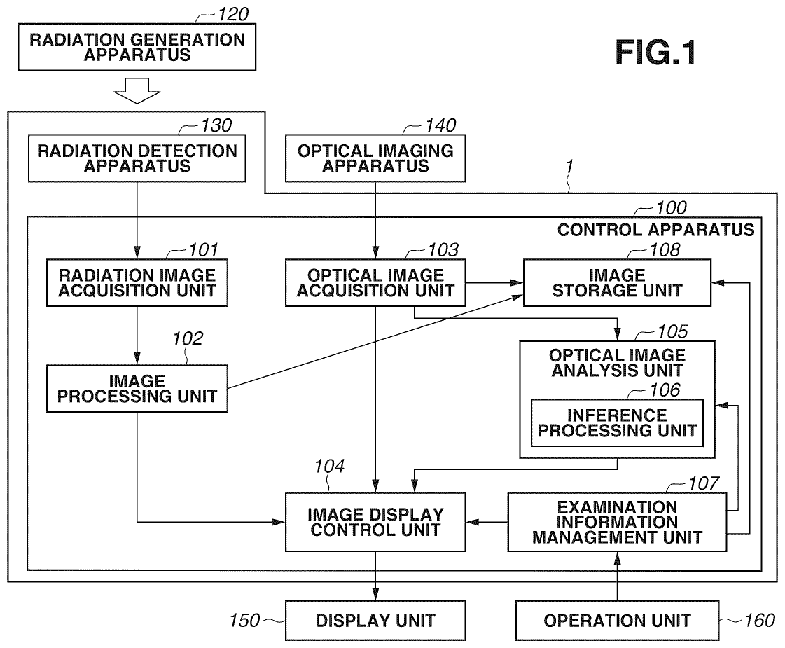

FIG. 1 is a block diagram illustrating an example of a basic configuration of a radiographic imaging system according to one or more embodiments. A radiographic imaging system 1 includes a control apparatus 100 serving as a radiographic imaging control apparatus, and at least one radiation detection apparatus 130. FIG. 1 further illustrates a radiation generation apparatus 120, an optical imaging apparatus 140, a display unit 150 serving as a display apparatus, and an operation unit 160. The control apparatus 100 controls radiographic imaging to be performed using the radiation detection apparatus 130 and the radiation generation apparatus 120.

The radiation detection apparatus 130 detects radiation emitted from the radiation generation apparatus 120 and having passed through a target, sample, or an examinee (not illustrated, and also referred to as a subject to be examined), and outputs image data corresponding to the radiation. The image data can also be referred to as a medical image or a radiation image. More specifically, the radiation detection apparatus 130 detects the radiation that has passed through the examinee, as an electric charge corresponding to a transmitted radiation amount. For example, the radiation detection apparatus 130 uses a direct conversion type sensor for converting radiation directly into an electric charge, such as an amorphous selenium (a-Se) sensor, or an indirect conversion type sensor that uses a scintillator such as a cesium iodide (CsI) sensor that converts radiation into visible light and a photoelectric conversion device such as an amorphous silicon (a-Si) sensor. Further, the radiation detection apparatus 130 generates image data by performing analog to digital (A/D) conversion on the detected charge, and outputs the image data to the control apparatus 100.

For example, the control apparatus 100 is connected with the radiation generation apparatus 120, the radiation detection apparatus 130, and the optical imaging apparatus 140 via a wired or wireless network or an exclusive line. The control apparatus 100 has a function of an application that operates on a computer. More specifically, the control apparatus 100 includes one or more processors and memories, and implements various functional units described below by causing the one or more processors to execute a program stored in the one or more memories. However, part or all of the functional units may be implemented by dedicated hardware, such as, but not limited to, one or more processors that operate to perform the one or more functions.

The control apparatus 100 controls a timing at which the radiation generation apparatus 120 generates radiation, and radiation imaging conditions, based on examination information received via the operation unit 160. A radiation image acquisition unit 101 functions as a second acquisition unit, controls timings at which the radiation detection apparatus 130 captures and outputs image data, and receives the generated image data. An image processing unit 102 performs image processing on the received image data, and the image display control unit 104 displays a processed image on the display unit 150. The image display control unit 104 provides a graphical user interface using the display unit 150, and receives an instruction from an operator via the operation unit 160. Examination information is selected based on the received input, and managed by an examination information management unit 107. As described above, the control apparatus 100 is a control apparatus for controlling radiographic imaging, and also controls operations of the radiation detection apparatus 130 and the radiation generation apparatus 120. Thus, the control apparatus 100 may also be referred to as a radiographic imaging control apparatus.

The control apparatus 100 controls a condition, a timing, a frame rate, and the like for acquisition of an optical image by the optical imaging apparatus 140. An optical image acquisition unit 103 functions as a first acquisition unit to acquire an optical image from the optical imaging apparatus 140. The image display control unit 104 adds information to be displayed on the display unit 150 serving as a display apparatus in addition to the optical image, and controls content to be displayed on the display unit 150. For example, the image display control unit 104 adds to the optical image an analysis result obtained by an optical image analysis unit 105 as a result of analyzing the optical image acquired by the optical image acquisition unit 103 serving as a first acquisition unit, and outputs the optical image with the analysis result added to the display unit 150 serving as a display apparatus. Details thereof will be described below. Further, the image display control unit 104 outputs and displays a radiation image acquired by the radiation image acquisition unit 101 to and on the display unit 150 serving as a display apparatus. The optical image analysis unit 105 analyzes a position, a kind, and an orientation of a target or a body part of a subject in the optical image acquired from the optical image acquisition unit 103, and a position and orientation of the target or subject in the optical image. Then, the optical image analysis unit 105 determines whether the analysis result matches the set content in the examination information acquired from the examination information management unit 107. An inference processing unit 106 that uses machine learning or the like may be used for the determination by the optical image analysis unit 105. An image storage unit 108 stores images acquired by the radiation image acquisition unit 101 or the optical image acquisition unit 103.

For example, a plurality of pieces of acquired examination information is displayed in a list format, and a user inputs an operation to select one of the plurality of pieces of examination information via the operation unit 160, so that the operation unit 160 sets the selected examination information as an examination target. In addition, a user may directly input the examination information via the operation unit 160. After the user selects the examination information, the operation unit 160 also receives, for example, an input for selecting imaging content to be an imaging target.

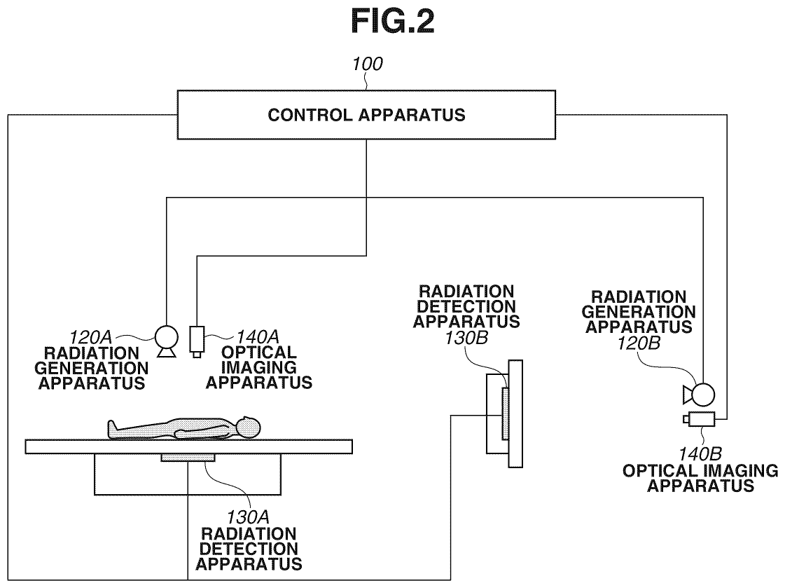

FIG. 2 is a diagram illustrating the radiographic imaging system 1 according to one or more embodiments. In an environment in which the radiographic imaging system 1 is actually used, there may be a plurality of radiation generation apparatuses 120, a plurality of radiation detection apparatuses 130, and a plurality of optical imaging apparatuses 140. In FIG. 2, an example in which the radiographic imaging system 1 includes two radiation generation apparatuses 120, two radiation detection apparatuses 130, and two optical imaging apparatuses 140 is illustrated. However, the numbers of the radiation generation apparatuses 120, the radiation detection apparatuses 130, and the optical imaging apparatuses 140 may be other than two.

Radiation generation apparatuses 120A and 120B each have identification information for uniquely identifying them. Further, radiation detection apparatuses 130A and 130B are each given identification information for uniquely identifying them. Further, optical imaging apparatuses 140A and 140B each have identification information for uniquely identifying them. These pieces of identification information are managed by the control apparatus 100. In addition, the optical imaging apparatus 140A is paired with the radiation generation apparatus 120A, and the optical imaging apparatus 140B is paired with the radiation generation apparatus 120B. The pairing information is also managed by the control apparatus 100.

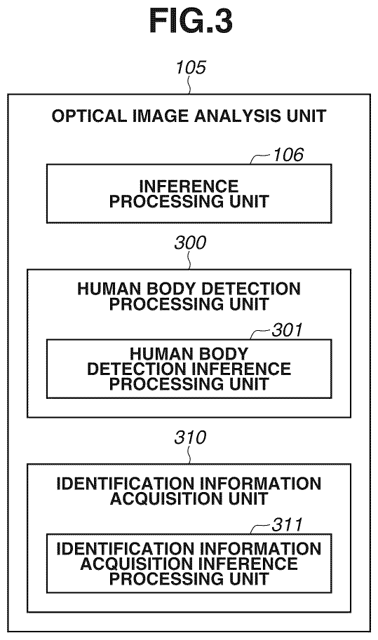

FIG. 3 is a block diagram illustrating a detailed configuration of the optical image analysis unit 105 according to one or more embodiments. In addition to the inference processing unit 106, the optical image analysis unit 105 includes a human body detection processing unit 300, a human body detection inference processing unit 301, an identification information acquisition unit 310, and an identification information acquisition inference processing unit 311.

The human body detection processing unit 300 performs human body detection processing to determine whether an examinee is present in an optical image acquired from the optical image acquisition unit 103, i.e., to determine whether a target or subject to be examined is present. In the human body detection processing by the detection processing unit 300, the human body detection processing unit 300 calculates a difference between frames, and determines that a human body is detected in a case where there is a difference between the frames. Further, the human body detection inference processing unit 301 may realize the human body detection processing by performing inference processing using a trained model trained by machine learning or deep learning. In this way, the human body detection processing unit 300 functions as a determination unit for determining whether a target or subject to be examined is present. Then, in a case where the human body detection processing unit 300 detects a human body in the optical image, the human body detection processing unit 300 notifies the control apparatus 100 of the detection of the human body. At this time, the control apparatus 100 acquires the identification information of the radiation generation apparatus 120 paired with the optical imaging apparatus 140 from the identification information of the optical imaging apparatus 140 from which the optical image acquisition unit 103 has acquired the optical image. For example, in the case of FIG. 2, a human body is detected by the optical imaging apparatus 140A, and the control apparatus 100 determines to use the radiation generation apparatus 120A because the optical imaging apparatus 140A is paired with the radiation generation apparatus 120A. The control apparatus 100 controls the radiation generation apparatus 120 of which the identification information corresponds to the acquired identification information to be in a state of being able to emit radiation. For example, in a case where the control apparatus 100 acquires the identification information of the radiation generation apparatus 120A, the control apparatus 100 makes or generates settings of imaging conditions and the like of the radiation generation apparatus 120A, and releases an irradiation lock of the radiation generation apparatus 120A to control the radiation generation apparatus 120A to be able to emit radiation when an operator presses a irradiation button (not illustrated). As described above, the control apparatus 100 serving as a radiographic imaging control apparatus makes or generates settings related to radiation generation in the radiation generation apparatus 120A that is to be used for radiographic imaging. Further, the control apparatus 100 desirably controls not to make or generate settings related to radiation generation in the radiation generation apparatus (in the case of FIG. 2, the radiation generation apparatus 120B) other than the radiation generation apparatus 120A to be used for the radiographic imaging.

The identification information acquisition unit 310 acquires the identification information of the radiation detection apparatus 130 to determine which radiation detection apparatus 130 is present in the optical image acquired from the optical image acquisition unit 103. The identification information acquisition inference processing unit 311 may acquire the identification information by performing inference processing using a trained model trained by machine learning or deep learning. As described above, the identification information acquisition unit 310 performs analysis related to the radiation detection apparatus 130 to determine the radiation detection apparatus 130 to be used for the radiographic imaging. Then, in a case where the identification information acquisition unit 310 acquires the identification information of the radiation detection apparatus 130, the identification information acquisition unit 310 notifies the control apparatus 100 of the acquired identification information of the radiation detection apparatus 130. The control apparatus 100 transmits a signal for shifting the state of the radiation detection apparatus 130 to be used for the examination to an imaging ready state based on the identification information of the radiation detection apparatus 130. For example, in the case of FIG. 2, the human body detection processing unit 300 determines that the radiation detection apparatus 130A is present in the optical image, and then the control apparatus 100 transmits to the radiation detection apparatus 130A a signal for shifting the state of the radiation detection apparatus 130A to an imaging ready state. As described above, the control apparatus 100 serving as a radiographic imaging control apparatus makes or generates settings related to radiation detection in the radiation detection apparatus 130A to be used for radiographic imaging.

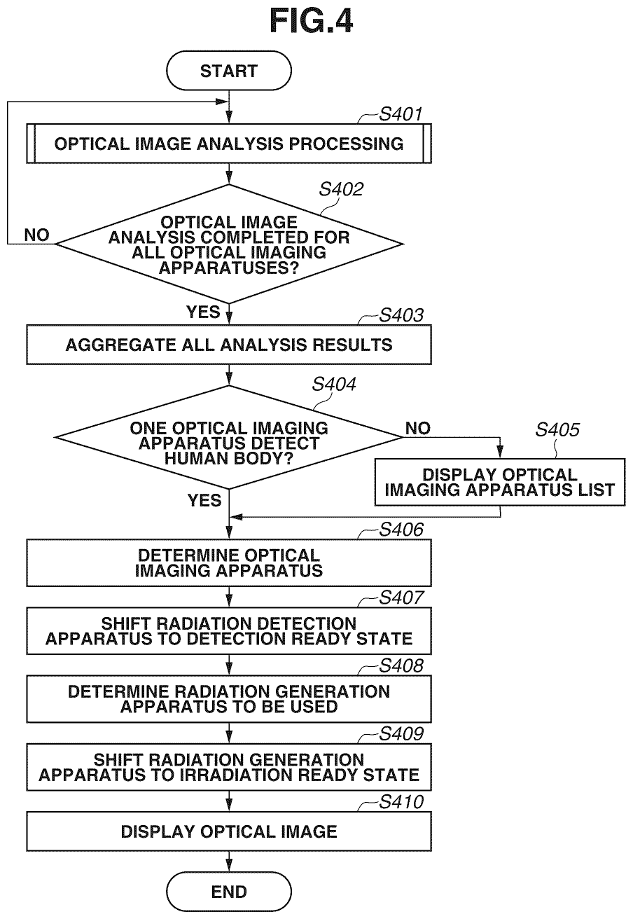

Next, with reference to a flowchart in FIG. 4, processing of switching the radiation generation apparatuses 120 using the optical imaging apparatuses 140 according to one or more embodiments will be described.

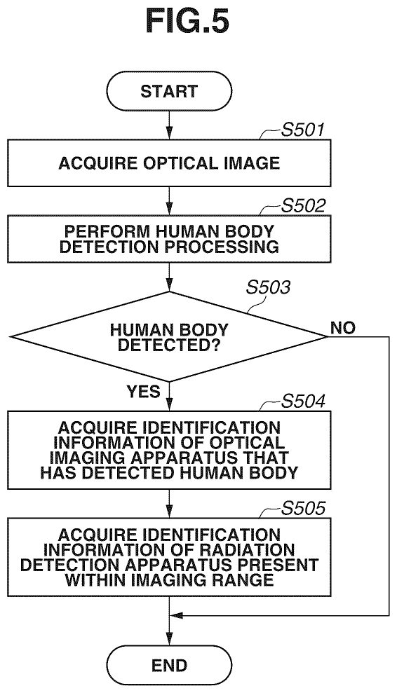

In step S401, the human body detection processing unit 300 performs optical image analysis processing illustrated in detail in FIG. 5.

In step S402, the control apparatus 100 determines whether the optical image analysis processing in step S401 is completed for all of the connected optical imaging apparatuses 140. In a case where the optical image analysis processing is completed for all of the connected optical imaging apparatuses 140 (YES in step S402), the processing proceeds to step S403. On the other hand, in a case where the optical image analysis processing is not completed for all of the connected optical imaging apparatuses 140 (NO in step S402), the processing returns to step S401.

In step S403, the control apparatus 100 aggregates all of the optical image analysis processing results, and checks whether there is the optical imaging apparatus 140 from which the control apparatus 100 is notified that the human body is detected, among the optical imaging apparatuses 140 connected to the control apparatus 100. Further, the control apparatus 100 checks identification information of a radiation detection apparatus 130 present within an imaging range of the notified optical imaging apparatus 140.

In step S404, the control apparatus 100 determines whether the number of optical imaging apparatuses 140 that have detected a human body is one, from the result in step S403. In a case where the number of optical imaging apparatuses 140 that have detected a human body is one (YES in step S404), the processing proceeds to step S406. On the other hand, the number of optical imaging apparatuses 140 that have detected a human body is 0, or 2 or more (NO in step S404), the processing proceeds to step S405.

In step S405, the control apparatus 100 displays, on the display unit 150, a list of the connected optical imaging apparatuses 140, or a list of the optical imaging apparatuses 140 that are determined to have detected a human body, and prompts, via the operation unit 160, the operator to select the optical imaging apparatus 140 the imaging range of which includes an examinee.

At this time, a possibility of the presence of a human body in each of the imaging ranges of the optical imaging apparatuses 140 may be displayed based on the probability of detecting a human body. In addition, instead of the possibility of the presence of a human body, the control apparatus 100 may determine and display a possibility of using each of the radiation generation apparatuses 120 as its probability. Further, the control apparatus 100 may display a message for notifying the operator that the determination cannot be automatically performed, together with the optical imaging apparatus list, and similarly, may display a message that the radiation generation apparatus 120 to be used cannot be determined. Further, the control apparatus 100 may display the optical images acquired from all of the optical imaging apparatuses 140 included in the optical imaging apparatus list, together with the optical imaging apparatus list. The image display control unit 104 controls the display contents to be output to the display unit 150. As described above, the image display control unit 104 can output the result of determination made by the optical image analysis unit 105 functioning as a determination unit to the display unit 150 serving as a display apparatus.

In step S406, the control apparatus 100 acquires the identification information of the optical imaging apparatus 140 that has detected a human body or the optical imaging apparatus 140 selected in step S405.

In step S407, the control apparatus 100 transmits to the radiation detection apparatus 130 a signal for shifting the state of the radiation detection apparatus 130 to a detection ready state, based on the identification information of the radiation detection apparatus 130 acquired in step S403. For example, in a case where the radiation detection apparatus 130 determined in step S403 is the radiation detection apparatus 130A, a main control circuit of the radiation detection apparatus 130A controls a bias power source to apply a bias voltage to a two-dimensional image sensor. Then, the radiation detection apparatus 130A performs initialization processing for reading out an image signal from an image pixel array by a drive circuit, in order to read out a dark current signal accumulated in the pixels. After the initialization processing is completed, the radiation detection apparatus 130A transmits to the control apparatus 100 state information indicating that the radiation detection apparatus 130A is ready for acquiring a radiation image.

In step S408, the control apparatus 100 acquires the identification information of the radiation generation apparatus 120 corresponding to the identification information of the optical imaging apparatus 140 acquired in step S406.

In step S409, the control apparatus 100 controls the radiation generation apparatus 120 having the identification information acquired in step S408 to be in an irradiation ready state.

In step S410, the image display control unit 104 acquires from the optical image acquisition unit 103 an optical image captured by the optical imaging apparatus 140 determined in step S406, and displays the acquired optical image on the display unit 150.

Next, with reference to a flowchart in FIG. 5, analysis processing of an optical image acquired from each of the optical imaging apparatuses 140 according to one or more embodiments will be described.

In step S501, the optical image acquisition unit 103 acquires optical images from the connected optical imaging apparatuses 140. For example, in a case where an optical image of the optical imaging apparatus 140A is analyzed, the optical image acquisition unit 103 acquires the optical image from the optical imaging apparatus 140A. The subsequent steps will be described using a case where the optical image of the optical imaging apparatus 140A is analyzed, as an example.

In step S502, the human body detection processing unit 300 performs human body detection processing on the optical image acquired from the optical imaging apparatus 140 in step S501. For example, in the human body detection processing, the human body detection processing unit 300 uses image processing for calculating a difference between frames of the optical image and determining that a human body is present in a case where there is a difference. Further, in the human body detection processing, inference processing may be performed by the inference processing unit 106 using machine learning or a trained model trained by machine learning or deep learning.

In step S503, the human body detection processing unit 300 determines whether a human body is detected, from the processing result in step S502. In a case where the human body detection processing unit 300 determines that a human body is detected (YES in step S503), the processing proceeds to step S504. On the other hand, in a case where the human body detection processing unit 300 determines that a human body is not detected (NO in step S503), the analysis processing is ended.

In step S504, the control apparatus 100 acquires the identification information of the optical imaging apparatus 140.

In step S505, the identification information acquisition unit 310 performs analysis processing on the optical image acquired from the optical image acquisition unit 103, and identifies a radiation detection apparatus 130 present within an imaging range of the optical imaging apparatus 140. For example, a bar-code is attached to the radiation detection apparatus 130A, and the identification information acquisition unit 310 identifies that the radiation detection apparatus 130A is present within the imaging range of the optical imaging apparatus 140A by reading the bar-code by the analysis processing.

As described above, according to one or more embodiments, the radiation generation apparatus 120 to be used for an examination can be automatically determined by determining whether an examinee is present within an imagining range of the optical imaging apparatus 140, from a result of image analysis processing performed by the optical imaging apparatus 140. In this way, the radiation generation apparatus 140 can be automatically switched depending on an examination room to be actually used or the state of an examinee, which makes it possible to improve the convenience.

In one or more embodiments, the human body detection processing is performed using the optical image acquired from the optical imaging apparatus 140, but it is merely an example. Any sensor can be used as long as the sensor can detect a human body, and the sensor for detecting a human body is not limited to the optical imaging apparatus 140. For example, the human body detection may be performed based on a change in distance information by using, for example, an infrared sensor or the like. In addition, the human body detection may be performed by using a sensor such as an ultrasonic sensor and a thermal sensor. Furthermore, the presence of an examinee may be detected and determined by detecting information attached to a human body (e.g., tag information of an integrated circuit (IC) tag mounted on the examinee). Further, a single sensor or a combination of a plurality of kinds of sensors may be used to detect a human body.

In one or more of the above-described embodiments, as illustrated in FIG. 2, the radiographic imaging system 1 including the plurality of radiation detection apparatuses (130A and 130B) is described, but it is not limited thereto, and, for example, there may be a case where only one radiation detection apparatus 130 is included in the radiographic imaging system 1. For example, in the radiographic imaging system 1 illustrated in FIG. 2, in a case where there is only the radiation detection apparatus 130B (i.e., there is no radiation detection apparatus 130A), the image display control unit 104 outputs to the display unit 150 a warning that the image capturing cannot be performed using the radiation generation apparatus 120A and causes the display unit 150 to display the warning. In this case, it is determined that there is an examinee in an optical image captured by the optical imaging apparatus 140A, but the presence of a radiation detection apparatus 130 is not detected in the optical image. Accordingly, taking the two analysis results into consideration, the image display control unit 104 outputs to the display unit 150 information (e.g., warning) indicating that a radiation generation apparatus 120 to be used for radiographic imaging cannot be determined and causes the display unit 150 to display the information, which makes it possible to prevent useless imaging from being performed. In this way, it may be desirable to determine a radiation generation apparatus to be used for radiographic imaging, using an analysis result regarding whether a target or subject to be examined is present and an analysis result regarding the presence of a radiation detection apparatus 130. Further, in this way, even in a case where there are fewer radiation detection apparatuses 130 than radiation generation apparatuses 120, for example, in a case where there is one radiation detection apparatus 130 for a plurality of radiation generation apparatuses 120, one or more features of the one or more embodiments may also be applied to the case and may achieve the above-described effect(s).

In one or more embodiments, the example in which the settings related to radiation generation are made or generated on the radiation generation apparatus 120 to be used for radiographic imaging is described, but the one or more embodiments of the present disclosure are not limited thereto. For example, only the settings related to the radiation generation apparatus 120 are to be made or generated without limiting to the settings related to radiation generation in one or more embodiments, and the settings include a setting for identifying the radiation generation apparatus 120 to be used on the display unit 150 in one or more embodiments. Further, in a case where an irradiation control apparatus for controlling the radiation generation apparatus 120 is separately provided when the settings are made or generated on the radiation generation apparatus 120 to be used, performing the settings on the irradiation control apparatus may be included in one or more embodiments and is within the scope of one or more techniques or features of the present disclosure.

According to one or more embodiments, for example, it is possible to determine a radiation generation apparatus 120 to be used, from information related to a target or subject to be examined acquired from a sensor or the like, to automatically set the radiation generation apparatus 120, and to reduce erroneous exposure to radiation. In this way, it is possible to make improvements, for example, simplification of an advance preparation.

In one or more embodiments, one or more differences, modifications, or variations from aforementioned one or more features of one or more embodiments that may be employed in one or more embodiments will be described.

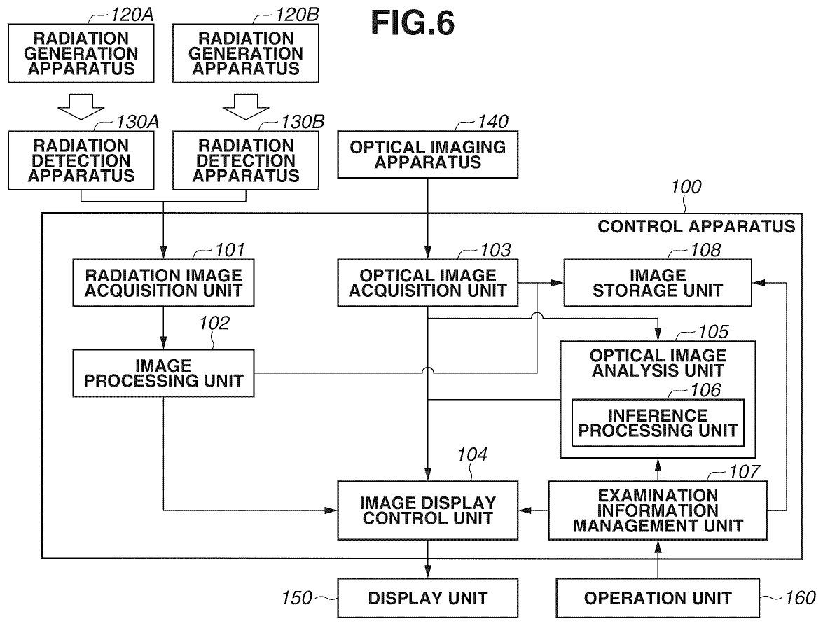

FIG. 6 is a block diagram illustrating an example of a configuration of a radiographic imaging system according to one or more embodiments. The radiographic imaging system 1 includes the control apparatus 100, the radiation generation apparatuses 120A and 120B, the radiation detection apparatuses 130A and 130B, the optical imaging apparatus 140, the display unit 150, and the operation unit 160.

The optical imaging apparatus 140 constantly captures an image of an entire examination room including an examinee, the radiation generation apparatuses 120A and 120B, and the radiation detection apparatuses 130A and 130B. More specifically, in one or more embodiments, a difference from aforementioned one or more embodiments in which the optical imaging apparatuses 140 and the radiation generation apparatuses 120 are associated with each other may be that the optical imaging apparatus 140 captures an image of the entire examination room. For example, the optical imaging apparatus 140 is connected with the control apparatus 100 via a wired or wireless network or an exclusive line. The optical image analysis unit 105 identifies a position of the examinee, and positions and orientations of the radiation generation apparatuses 120 and the radiation detection apparatuses 130. The optical image analysis unit 105 may perform inference processing using a trained model trained by machine learning or deep learning to identify the positions and the orientations.

The control apparatus 100 determines which radiation generation apparatus 120 is to be used for an examination from among the connected radiation generation apparatuses 120 (i.e., 120A and 120B), based on a result of analysis performed by the optical image analysis unit 105. For example, the control apparatus 100 determines that the radiation generation apparatus 120A is to be used for the examination from the information about the position and orientation of the radiation generation apparatus 120A, and position information about the examinee.

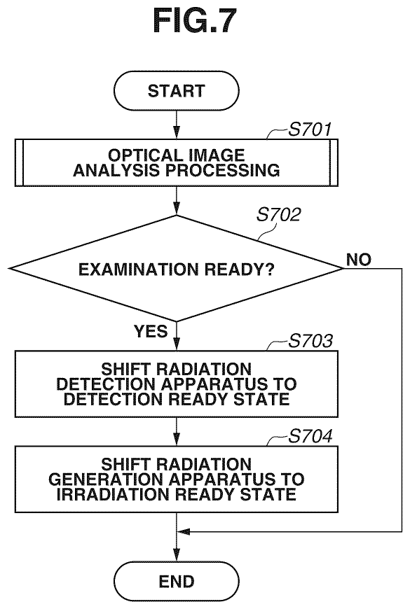

Next, with reference to a flowchart in FIG. 7, processing of switching radiation generation apparatuses 120 using the optical imaging apparatus 140 according to one or more embodiments will be described.

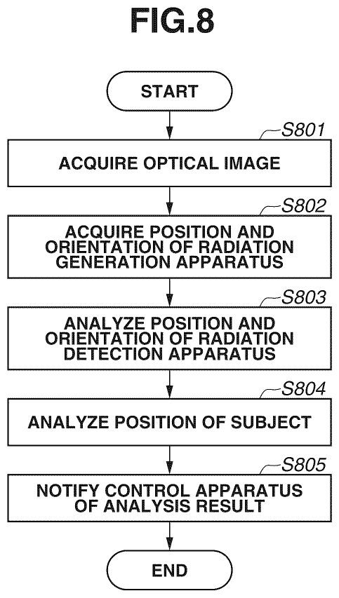

In step S701, the optical image analysis unit 105 performs optical image analysis processing illustrated in detail in FIG. 8.

In step S702, the control apparatus 100 determines whether the examinee and the radiation detection apparatus 130 are present in a direction in which the radiation generation apparatus 120 emits radiation and determines whether the preparations for the examination are completed, based on the analysis result in step S701. For example, from the analysis result in step S701, in a case where the control apparatus 100 determines that the examinee is present in the direction in which the radiation generation apparatus 120 emits radiation but the radiation detection apparatus 130 is not present, the control apparatus 100 determines that the preparations for the examination are not completed. In a case where the preparations for the examination are completed (YES in step S702), the processing proceeds to step S703. On the other hand, in a case where the preparations for the examination are not completed (NO in step S702), the processing may be ended, or a warning indicating that no radiation detection apparatus 130 is present may be displayed on the display unit 150.

In step S703, the control apparatus 100 transmits to the radiation detection apparatus 130 a signal for shifting the state of the radiation detection apparatus 130 to a detection ready state, based on the identification information of the radiation detection apparatus 130 acquired in step S701. For example, in a case where the radiation detection apparatus 130 determined in step S701 is the radiation detection apparatus 130A, a main control circuit of the radiation detection apparatus 130A controls a bias power source to apply a bias voltage to a two-dimensional image sensor. Then, the radiation detection apparatus 130A performs initialization processing for reading out an image signal from an image pixel array by a drive circuit, in order to read out a dark current signal accumulated in the pixels. After the initialization processing is completed, the radiation detection apparatus 130A transmits to the control apparatus 100 state information indicating that the radiation detection apparatus 130A is ready for acquiring a radiation image.

In step S704, the control apparatus 100 controls the radiation generation apparatus 120 determined to have completed the preparations for the examination in step S702 to be in a state of being able to emit radiation. For example, in a case where the control apparatus 100 determines to perform the examination using the radiation generation apparatus 120A, the control apparatus 100 makes or generates settings of imaging conditions and the like in the radiation generation apparatus 120A, releases a irradiation lock of the radiation generation apparatus 120A, and controls the radiation generation apparatus 120A to be in the state of being able to emit radiation when the operator presses a irradiation button (not illustrated).

Next, with reference to a flowchart in FIG. 8, analysis processing of an optical image acquired from the optical imaging apparatus 140 according to one or more embodiments will be described.

In step S801, the optical image acquisition unit 103 acquires an optical image from the optical imaging apparatus 140.

In step S802, the optical image analysis unit 105 analyzes the position and orientation of the radiation generation apparatus 120 present within an imaging range, using the optical image acquired in step S801. Further, the optical image analysis unit 105 acquires the identification information of the radiation generation apparatus 120 present within the imaging range. The analysis of the position and orientation of the radiation generation apparatus 120, and the acquisition of the identification information of the radiation generation apparatus 120 may be performed using, for example, inference processing using a trained model trained by machine learning or deep learning.

In step S803, the optical image analysis unit 105 analyzes the position and orientation of the radiation detection apparatus 130 present within the imaging range, using the optical image acquired in step S801. Further, the optical image analysis unit 105 acquires the identification information of the radiation detection apparatus 130 present within the imaging range. The analysis of the position and orientation of the radiation detection apparatus 130, and the acquisition of the identification information of the radiation detection apparatus 130 may be performed using, for example, inference processing using a trained model trained by machine learning or deep learning.

In step S804, the optical image analysis unit 105 analyzes the position of the examinee present within the imaging range, using the optical image acquired in step S801. The position analysis of the examinee may be performed using, for example, inference processing using a trained model trained by machine learning or deep learning.

In step S805, the optical image analysis unit 105 notifies the control apparatus 100 of the results of analysis in steps S802 to S804.

As described above, according to one or more embodiments, it is possible to automatically determine the radiation generation apparatus 120 to be used for the examination based on the information about the positions and orientations of the apparatuses in the examination room and the position information about the examinee, by performing the image analysis processing on the image captured by the optical imaging apparatus 140 that is capturing the entire examination room. In this way, the radiation generation apparatus 140 can be automatically switched depending on a state of an examination room to be actually used or a state of an examinee, which makes it possible to improve the convenience.

In one or more embodiments, the position of the examinee, and the position and orientation of each apparatus may be detected using the optical image acquired from the optical imaging apparatus 140, but this is merely an example and such one or more embodiments are not limited thereto. Any sensor can be used as long as the sensor can detect the position and orientation, and it is not limited to the optical imaging apparatus 140. Further, a single sensor can be used or a plurality of kinds of sensors may be used in combination. For example, the position information about the examinee and each apparatus may be acquired by using the radio-frequency identification (RFID), and the orientation information about each apparatus may be acquired by using an acceleration sensor.

Further, to improve the accuracy in the determination of the radiation generation apparatus 120 to be used for the examination, the optical imaging apparatus 140 described in one or more embodiments and the human body detection sensor described in one or more embodiments may be used in combination. For example, the position information about the examinee and each apparatus and the orientation information about each apparatus, acquired from the optical image of the optical imaging apparatus 140 that is capturing the entire examination room, and the human body detection information acquired from the optical imaging apparatus 140 paired with the radiation generation apparatus 120, may be comprehensively analyzed.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to one or more embodiments, it is to be understood that the scope of the present disclosure is not limited to the disclosed embodiments. The scope of the following claims is to be accorded the broadest interpretation so as to encompass all such modifications, equivalent structures, and functions.

What is claimed is:

1. A radiographic imaging control apparatus that operates or is configured to control radiographic imaging, the radiographic imaging being performed by emitting radiation to at least one radiation detection apparatus using at least one radiation generation apparatus from among a plurality of radiation generation apparatuses, the radiographic imaging control apparatus comprising:
a first acquisition unit that operates or is configured to acquire information about the radiographic imaging;
an analysis unit that operates or is configured to analyze the information acquired by the first acquisition unit to determine at least whether a target or subject to be examined is present; and
a determination unit that operates or is configured to determine a radiation generation apparatus to be used for the radiographic imaging among the plurality of radiation generation apparatuses, based on a result of analysis performed by the analysis unit,
wherein the radiographic imaging control apparatus makes or generates a setting related to the radiation generation apparatus determined by the determination unit to be used for the radiographic imaging.

2. The radiographic imaging control apparatus according to claim 1, wherein one or more processors of the radiographic imaging control apparatus operate to perform the setting related to the radiation generation apparatus on the radiation generation apparatus determined by the determination unit to be used for the radiographic imaging.

3. The radiographic imaging control apparatus according to claim 1,
wherein the analysis unit further performs analysis related to the radiation detection apparatus on the information acquired by the first acquisition unit, and
wherein the determination unit determines the radiation generation apparatus to be used for the radiographic imaging among the plurality of radiation generation apparatuses, based also on a result of the analysis related to the radiation detection apparatus performed by the analysis unit.

4. The radiographic imaging control apparatus according to claim 3,
wherein, in a case where a plurality of radiation detection apparatuses is present, the determination unit determines a radiation detection apparatus to be used for the radiographic imaging among the plurality of radiation detection apparatuses, based on the result of the analysis related to the radiation detection apparatus performed by the analysis unit, and
wherein the radiographic imaging control apparatus makes or generates a setting related to radiation detection on the radiation detection apparatus determined by the determination unit to be used for the radiographic imaging.

5. The radiographic imaging control apparatus according to claim 4, wherein one or more processors of the radiographic imaging control apparatus operate to perform the setting related to the radiation detection on the radiation detection apparatus determined by the determination unit to be used for the radiographic imaging.

6. The radiographic imaging control apparatus according to claim 1, wherein the radiographic imaging control apparatus does not make or generate a setting related to a radiation generation apparatus other than the radiation generation apparatus to be used for the radiographic imaging among the plurality of radiation generation apparatuses.

7. The radiographic imaging control apparatus according to claim 1, further comprising:
a second acquisition unit that operates or is configured to acquire information related to a radiation image from the radiation detection apparatus; and
a display control unit that operates or is configured to output the information acquired by the second acquisition unit to a display apparatus.

8. The radiographic imaging control apparatus according to claim 7, wherein the display control unit operates or is configured to output a result of determination made by the determination unit to the display apparatus.

9. The radiographic imaging control apparatus according to claim 8, wherein, in a case where the determination unit is not able to determine a radiation generation apparatus to be used for the radiographic imaging, the display control unit outputs to the display apparatus information indicating that the determination unit is not able to determine a radiation generation apparatus to be used for the radiographic imaging.

10. The radiographic imaging control apparatus according to claim 8, wherein the determination unit determines a probability of the radiation generation apparatus to be used for the radiographic imaging among the plurality of radiation generation apparatuses, based on the result of the analysis performed by the analysis unit.

11. A radiographic imaging system, comprising:
a radiation detection apparatus; and
a radiographic imaging control apparatus that operates or is configured to control radiographic imaging, the radiographic imaging being performed by emitting radiation to at least one radiation detection apparatus using at least one radiation generation apparatus from among a plurality of radiation generation apparatuses, the radiographic imaging control apparatus comprising:
a first acquisition unit that operates or is configured to acquire information about the radiographic imaging;
an analysis unit that operates or is configured to analyze the information acquired by the first acquisition unit to determine at least whether a target or subject to be examined is present; and
a determination unit that operates or is configured to determine a radiation generation apparatus to be used for the radiographic imaging among the plurality of radiation generation apparatuses, based on a result of analysis performed by the analysis unit,
wherein the radiographic imaging control apparatus makes or generates a setting related to the radiation generation apparatus determined by the determination unit to be used for the radiographic imaging.
